(19) United States
(12) Patent Application Publication
(10) Pub. No.: US 2009/0158595 A1
(43) Pub. Date: Jun. 25, 2009

(54) Microemulsion and Shaving Apparatus Comprising a Microemulsion

(76) Inventors: David Acher, Hull (GB); Paul Ellis, Hull (GB); Tracey Heeson, Hull (GB)

Correspondence Address: TRASKBRITT, P.C., P.O. BOX 2550, SALT LAKE CITY, UT, 84110, US

(21) Appl. No.: 12/083,470

(22) PCT Filed: Oct. 16, 2006
(86) PCT No.: PCT/GB2006/003814

§ 371 Date: Sep. 29, 2008

(30) Foreign Application Priority Data
Oct. 14, 2005 (GB) 0520931.7

(51) Int. Cl. B26B 19/38; A61K 8/89; A61Q 9/04; A45D 27/00

(52) U.S. Cl. 30/41; 424/70.12; 30/541

(57) ABSTRACT

A microemulsion comprising an oil phase, an aqueous phase, a surfactant, and a hair modification active. The microemulsion is suitable for being impregnated therein or deposited thereon a substrate.

The present invention relates to a microemulsion comprising a hair modification active. The present invention also relates to a composite containing the microemulsion and a shaving apparatus including the microemulsion.

Various methods for removing hair from dermal surfaces are known. For example, hair may-be removed from the skin using a depilatory composition. Such compositions typically take the form of creams or lotions and contain a depilatory active that is capable of degrading hair keratin.

In an alternative method, hair is removed from dermal surfaces using a razor. In a typical shaving method, the razor is moved across the surface of the skin; hair on the skin's surface is cut as it comes into contact with the razor blade(s). Optionally, a shaving aid, such as shaving cream, may be applied to the skin prior to shaving.

Although shaving provides an effective method of hair removal, the hair that is cut using a razor blade tends to have a hard and bristly appearance and feel. It is therefore among the objects of the embodiments of the present invention to develop a composition that can be used to provide hair with an improved appearance and feel, for example, after cutting.

According to a first aspect of the present invention, there is provided a microemulsion comprising
an oil phase,
an aqueous phase,
a surfactant, and
a hair modification active.

In this application the term hair modification covers both depilation (whereby the keratin present in hair is substantially broken down therefore effecting removal of the hair) and hair regrowth inhibition whereby an active is applied to a hair shaft or follicle thereby substantially reducing the hair regrowth. Hair modification is not intended to cover change the color of hair by, for example, dying.

The hair modification active may be a depilatory active or an active which substantially inhibits the regrowth of hair (commonly known as a hair regrowth inhibitor or hair regrowth inhibition active).

Preferably, the microemulsion includes a co-surfactant in addition to the surfactant.

When the microemulsion is applied onto the skin, for example, after shaving, it advantageously acts on the freshly cut hairs to provide the hairs with a more rounded and/or soft appearance.

The term “microemulsion” is used herein to describe a liquid system comprising droplets or swollen micelles having oily or aqueous centres. These droplets are typically less than 200 nm in size, preferably less than 150 nm in size, more preferably less than 100 nm in size. The droplets of the dispersed phase may be 5 to 90 nm, more preferably from 10 to 80 nm, even more preferably 15 to 75 nm, for. example 30 to 70 nm in size.

Without wishing to be bound by any theory, the droplets of the microemulsion are believed to be surrounded by an interfacial film in which the surfactant and co-surfactant alternate. The interfacial tension between the oil phase and the water phase is generally low due to the surfactant and optional co-surfactant in the system. Typically, the interfacial tension between the oil phase and aqueous phase is from 0.1 to 50 mN/m, preferably 1 to 40 mN/m, preferably 10 to 30 mN/m.

The microemulsions of the present invention may have a discontinuous or bi-continuous structure, depending on the relative concentrations of oil, water and surfactant in the system. In one embodiment, the microemulsion comprises micro-areas of the aqueous phase that are randomly organized and separated from micro-areas of the oil phase by the interfacial film.

Preferably, the microemulsion is a singly optically isotropic liquid solution. Preferably, the microemulsion is thermodynamically stable.

The hair modification active may be present in the oil phase and/or the aqueous phase of the microemulsion.

It is believed that many hair modification actives may have both depilatory and hair re-growth inhibition properties. It is further believed that the amount of hair modification active present and/or the pH in the hair modification composition determines whether the composition acts as a hair re-growth inhibition composition or a depilatory composition.

Accordingly, the composition when containing a hair modification active, may act as a depilatory composition or a hair re-growth inhibition composition. The hair modification active may therefore be selected from, but not restricted to a sulphur compound, such as potassium thioglycolate, sodium thioglycolate, ammonium thioglycolate, dithioetythritol, thioglycerol, thioglycol, thioxanthine, thipsalicylcic acid, N-acetyl-L-cysteine, lipic acid, NaHSO₃, Li₂S, Na₂S, K₂S, MgS, CaS, SrS, BaS, (NH₄) ₂S, sodium dihydrolipoate 6, 8-dithiooctanoate, sodium 6,8-dithiooctanoate, salts of hydrogen sulphide (e.g. NaSH or KSH), thioglycolic acid, thioglycerol, 2-mercaptopropionic acid, 3-mercaptropropionic acid, mercaptoethanol, dithioerythritol (DTE), gluatothione (reduced form), thiomalic acid, calcium thioglycolate, guanidine thioglycolate, glyceryl monothioglycolate, monoethanolamine thioglycolate, monoethanolamine thioglycolic acid, diammonium dithiodiglycolate, ammonium thiolactate, monoethanolamine thiolactate, thioglycolamide, homo-cysteine, cysteine, acetyl cysteine, glutathione, dithiothreitol, dihydrolipoic acid, 1, 3-dithiopropanol, thioglycolamide, glycerolmonothioglycolate, thioglycolhydrazine, keratinase, hydrazine sulphate, hydrazine disulphate triisocyanate, guanidine thioglycolate, thiosalysilic acid, calcium thioglycolate, cysteamine, Xyleine, lipoic acid, sodium dihydrolipoate, thiolactic acid, thiopropionic acid, 2 thiolhistidene, 6 mercaptopurine, dimercaptosuccinic acid, thiophenol, 4-mthoxythiophenol, 4 bromothiophenol, benzyl mercaptan, 2 mercaptobenzothiazole, bromhexine, carbocysteine, domiodol, erdosteine, letosteine, lysozyme, mecysteine hydrochloride, mesna, sobrerol, stepronin, tiopronin, tyloxapol, nor-dihydroguaiaretic acid (NDGA), resveratrol and/or Bowman Birk inhibitor (BBI-AV). It is, of course, envisaged that blends of more than one hair modification active may be used

Preferred hair modification actives are thioglycolates, or their precursor thioglycolic acid; glycerol monothioglycolate, cysteamine, acetyl cysteine (N-acetyl-L-Cysteine) and BBI-AV An example of a suitable thioglycolate is potassium thioglycolate, which may be produced by mixing thioglycolic acid with a neutralizing source of potassium hydroxide.

Preferred hair modification actives are thioglycolates, or their precursor thioglycolic acid; glycerol monothioglycolate, cysteamine and acetyl cysteine (N-acetyl-L-Cysteine). Most preferably, the hair modification active is acetyl cysteine (N-acetyl-L-Cysteine), and/or cysteamine.

The microemulsion may comprise 0.01 to 10 weight %, preferably 0.05 to 5 weight %, more preferably 0.1 to 2 weight % of the hair modification active. In one embodiment, from 0.3 to 1 weight % hair modification active is present. Preferably, the composition includes at least one skin enhancing active. The skin enhancing active is preferably a compound which is typically used in skin care products. Such agents may reduce wrinkles, acne scarring, substantially reduce in-growing hairs, and/or chemically exfoliating the skin.

The at least one skin enhancing active is preferably a chemical exfoliating active such as an alpha hydroxyl acids (AHAs), such as salicylic acid, glycolic acid, lactic acid, malic acid, citric acid, tartaric acid, trichloracetic acid, or a Polyhydroxyacid (PHA), such as gluconalactone.

The chemical exfoliating active is typically present in an amount in the range 0.05 to 3.0 % by weight of the composition, preferably 0.1 to 2.0% by weight of the composition, such as 0.5 to 1.5% by weight. The oil phase of the microemulsion may include oil(s) in an amount of 30 to 90 weight %, preferably 50 to 80 weight %, more preferably 55 to 70 weight % of the microemulsion.

Suitable oils include polysiloxanes, silicones, mineral oils and essential oils. Mixtures of two or more oils may be employed.

Preferably, the oil phase comprises a polysiloxane. Examples of suitable polysiloxanes include diquaternary polydimethylsiloxane, cyclohexasiloxane and/or cyclomethicone. In a preferred embodiment, a diquaternary polydimethylsiloxane is used in combination with at least one of cyclohexasiloxane and/or cyclomethicone. In one example of the microemulsion, 20 to 40 weight % diquaternary polydimethylsiloxane and 20 to 40 weight % of cyclohexasiloxane and/or cyclomethicone.

The microemulsion of the present invention includes a surfactant. The surfactant may be present in an amount of 1 to 30 weight %, preferably 2 to 20 weight %, for example 5 to 10 weight %. Any suitable surfactant may be employed. For example, the surfactant may be a non-ionic, anionic, cationic or amphoteric surfactant. Preferably, however, the surfactant is non-ionic. Examples of suitable surfactants include non-ionic ester surfactants, such as glyceryl esters and their derivatives.

Preferably, the surfactant is a polyglycolised glyceride. The polyglycolised glyceride may be present in an amount of 1 to 30 weight %, preferably 2 to 20 weight %, more preferably 5 to 10 weight %, for example 6 to 8 weight % of the microemulsion. The polyglycolised glyceride may have 8 to 20 carbon atoms, for example, 8 to 10 carbon atoms. In one embodiment, the surfactant is a polyethylene glycol derivative of a triglyceride of capric acid or caprylic acid. In a preferred embodiment PEG-8 capric/caprylic glycerides are used. This surfactant may be present in an amount of 6 to 8 weight % of the microemulsion.

The microemulsion preferably includes a co-surfactant. The co-surfactant is preferably different from the surfactant. The co-surfactant may be present in an amount of 1 to 30 weight %, preferably 2 to 20 weight %, more preferably 5 to 10 weight %, even more preferably 6 to 8 weight %. Any suitable co-surfactant may be employed. For example, the surfactant may be a non-ionic, anionic, cationic or amphoteric surfactant. Preferably, however, the co-surfactant is non-ionic. In one embodiment, a non-ionic ester surfactant is used. Alcohol surfactants may also be suitable.

In a preferred embodiment, the co-surfactant is a fatty acid ester and/or a polyglycerol. An example of a suitable surfactant is triglyceryl diisostearate. Triglyceryl diisostearate may be present in an amount of 6 to 8 weight % of the microemulsion.

Where a surfactant and co-surfactant are present in the microemulsion, the ratio of surfactant to co-surfactant in the microemulsion may be 0.05:1 to 10:1, preferably 0.2:1 to 5:1, more preferably 0.5:1 to 4:1. In one embodiment, the surfactant and co-surfactant are present in a ratio of 1:1.

The microemulsion may include an additive that improves the efficacy of the co-surfactant. Examples of such additives include glycol ethers. A glycol ether having 4 to 20 carbon atoms, preferably 4 to 12 carbon atoms, for example, 6 to 8 carbon atoms may be employed. In a preferred embodiment, 2-(2-ethoxyethoxy)ethanol is used. Such additives may be present in an amount of 1 to 20 weight %, preferably 2 to 15 weight %, for example 6 to 10 weight % of the microemulsion.

Optionally, the microemulsion includes an accelerator that will accelerate the keratin degradation reaction such as urea, thiourea, dimethyl, isosorbide (DMI), ethoxydiglycol (Transcutol) or methyl propyl diol (MP diol). Preferably the accelerator is urea. The microemulsion according to the invention preferably comprises from 5 to 15% weight of an accelerator relative to the weight of the microemulsion, more preferably from 6 to 10 wt %.

The microemulsion may also include a fragrance. The fragrance may be present in an amount of 0.01 to 5 weight %, preferably 0.1 to 2 weight %, for example 0.7 weight %.

The microemulsion may optionally include a dye. The dye may be present in an amount of 0.001 to 5 weight %, for example 0.001 to 1 weight % of the solid depilatory composition.

The microemulsion may optionally include a skin conditioning active. Such actives may be present in an amount of from 0.01 to 10 weight % of the microemulsion. Preferably, the skin conditioning active is present in an amount of 0.01 to 5 weight %, for example, 0.1 to 1 weight %. The skin conditioning active may be silicone or a polysiloxane.

The microemulsion may optionally include a vitamins, such as Vitamin E. Vitamins may be present in an amount of from 0.01 to 10 weight % of the microemulsion. Preferably, the Vitamin is present in an amount of 0.01 to 5 weight %, for example, 0.1 to 1 weight %.

The microemulsion may also include a preservative. The preservative may be present in an amount of 0.01 to 5 weight %, for example, 0.1 to 1 weight %.

As described above, the oil phase may form 30 to 90 weight %, preferably 50 to 80 weight %, more preferably 55 to 70 weight % of the microemulsion. Water may form 5 to 40 weight %, preferably 10 to 30 weight %, more preferably 15 to 20 weight % of the microemulsion.

The ratio of the oil phase to the aqueous phase in the microemulsion may be 0.5:1 to 20:1, preferably 2:1 to 6:1, more preferably 4:1 to 5:1.

The PH of the microemulsion is preferably in the range 4 to 13, a further preferably 5 to 10, such as 5.5 to 8.5.

The present invention further provides a method of modifying hair comprising:
applying the microemulsion according to the first aspect of the invention onto the skin, optionally, in the presence of water,
leaving the microemulsion on the skin for a residence time sufficient to modify hairs on the skin, and
removing the microemulsion and depilated hairs from the skin.

Preferably the residence time is less than 15 minutes, more preferably not more than 10 minutes, even more preferably not more than 8 minutes. Preferably the residence time is at least 0.5 seconds, more preferably at least 1 seconds, even more preferably at least 5 seconds.

The microemulsion may be removed from the skin by any means, for example using a sponge, spatula or scraper device. The microemulsion may also be removed by rinsing.

The microemulsion of the present invention may be employed as a shaving aid. For example, the microemulsion of the present invention may be applied to the surface of the skin prior to or, preferably, after shaving. The microemulsion may be applied to wet or dry skin.

By applying the microemulsion onto the skin, for example, after shaving, the microemulsion may act on the freshly cut hairs to provide the hairs with a rounded and/or soft appearance.

The microemulsion may be stored in or adjacent the shaving head of a razor and at least partially released when the shaving head comes into contact with, for example, a dermal surface.

According to a further aspect of the present invention, there is provided a composite for use in a shaving apparatus, which composite comprises
a support, and
the microemulsion of the present invention deposited or impregnated on the support.

When the loaded support is contacted with the skin, it releases at least some of the deposited microemulsion onto the skin's surface.

The composite may be coupled to a shaving apparatus, for example, adjacent the head of the razor. In a preferred embodiment, the composite is positioned adjacent one or more blades of the shaving head. The composite may form the glide strip of a shaving apparatus. In this way, the microemulsion may be released when the shaving apparatus is in use.

Preferably, the microemulsion is impregnated into the support. When the supported is freshly loaded, the weight ratio of microemulsion to support may be 10:1 to 1:20, preferably 2:1 to 1:5, for example, 1:1 to 1:4

The support may be formed of any suitable material. Preferably, the support is formed at least in part of a polymer. Suitable polymers include PTFE. In one embodiment, the support comprises a coating of, for example, a polymer. Suitable coatings include coatings formed of PTFE.

However, it is also envisaged that the support may include any suitable material that permits the microemulsion to be deposited thereon or impregnated therein. Accordingly, the support may include a porous material (such as synthetic or natural sponge), a fibrous material (synthetic or natural), leather (such as chamois leather) or a cellulose based material.

The support may optionally include a dye.

According to a further aspect of the present invention, there is provided a shaving apparatus comprising

a shaving head, and a support member having deposited thereon,
or impregnated therein, a microemulsion containing a depilatory active.

The microemulsionn is preferably a microemulsion according to the first aspect of the present invention.

According to a further aspect of the present invention, there is provided a shaving apparatus comprising
a shaving head, having arranged thereon the composite of the present invention, said composite being configured to release at least some of the microemulsion from the shaving apparatus when the apparatus is in use.

The shaving head typically includes one or more blades, the composite being positioned adjacent the one or more blades.

The shaving head is preferably connected to, or removably attached to, a razor handle.

According to a further aspect of the present invention, there is provided a shaving apparatus comprising
a shaving head, and
means containing the microemulsion of the present invention, said means being configured to release at least some of the microemulsion from the shaving apparatus when the apparatus is in use.

The following Examples further illustrate the present invention.

### EXAMPLE 1

A microemulsion was prepared from the following ingredients:

| | Component | % w/w |
|---|---|---|
| (i) | PEG-8 Caprylic/Capric Glycerides | 7.0 |
| (ii) | Polyglyceryl-3 Diisotearate | 7.0 |
| (iii) | Diquaternary polydimethylsiloxane | 26.6 |
| (iv) | Cyclohexasiloxane or Cyclomethicone | 30.1 |
| (v) | Ethoxydiglycol | 7.4 |
| (vi) | Preservative | 0.60 |
| (Vii) | N-acetyl-l-cysteine | 0.92 |
| (Viii) | Water | 20.38 |

A solution of 3% N-acetyl-1-cysteine distilled water was prepared. Components (i) to (vi) were then mixed together at room temperature in sequential order. The N-Acetyl-1-cysteine solution was then added to the mixture to form the microemulsion. Sufficient N-Acetyl-1-cysteine solution was added to ensure that the concentration of N-Acetyl-1-cysteine in the microemulsion was 0.52% N-acetyl-l-cysteine. The concentration. of distilled water in the microemulsion was 16.73%.

The microemulsion was used to impregnate a PTFE (Teflon™) support. The support was immersed in the microemulsion for 24 hours. The impregnated support was then coupled to a razor using an adhesive.

### EXAMPLE 2

A microemulsion was prepared from the following ingredients:

| | Component | % w/w |
|---|---|---|
| (i) | PEG-8 Caprylic/Capric Glycerides | 7.0 |
| (ii) | Polyglyceryl-3 Diisotearate | 7.0 |
| (iii) | Diquaternary polydimethylsiloxane | 26.6 |
| (iv) | Cyclohexasiloxane or Cyclomethicone | 30.1 |
| (v) | Ethoxydiglycol | 7.4 |
| (vi) | Preservative | 0.60 |
| (Vii) | NAC | 0.92 |
| (viii) | Salicylic acid | 1.0 |
| (ix) | Water | 19.38 |

A solution of 3% N-acetyl-1-cysteine distilled water was prepared; the salicylic acid is prepared into the same solution as the NAC. Components (i) to (vi) were then mixed together at room temperature in sequential order. The N-Acetyl-1-cysteine and salicylic solution was then added to the mixture to form the microemulsion. Sufficient N-Acetyl-1-cysteine solution was added to ensure that the concentration of N-Acetyl-1-cysteine in the microemulsion was 0.52% N-acetyl-1-cysteine. The concentration of distilled water in the microemulsion was 16.73%.

The microemulsion was used to impregnate a PTFE (Teflon™) support. The support was immersed in the microemulsion for 24 hours. The impregnated support was then coupled to a razor using an adhesive.

### EXAMPLE 3

A microemulsion was prepared from the following ingredients:

| | Component | % w/w |
|---|---|---|
| (i) | PEG-8 Caprylic/Capric Glycerides | 7.0 |
| (ii) | Polyglyceryl-3 Diisotearate | 7.0 |
| (iii) | Diquaternary polydimethylsiloxane | 26.6 |
| (iv) | Cyclohexasiloxane or Cyclomethicone | 30.1 |
| (v) | Ethoxydiglycol | 7.4 |
| (vi) | Preservative | 0.60 |
| (Vii) | NAC | 0.92 |
| (viii) | glycolic acid | 5.5 |
| (ix) | Water | 14.88 |

A solution of 3% N-acetyl-1-cysteine distilled water was prepared. Components (i) to (vi) were then mixed together at room temperature in sequential order. The N-Acetyl-1-cysteine solution was then added to the mixture to form the microemulsion. Sufficient N-Acetyl-1-cysteine solution was added to ensure that the concentration of N-Acetyl-1-cysteine in the microemulsion was 0.52% N-acetyl-1-cysteine. The concentration of distilled water in the microemulsion was 16.73%. The glycolic acid is added directly to the microemulsion as it is already a solution.

The microemulsion was used to impregnate a PTFE (Teflon™) support. The support was immersed in the microemulsion for 24 hours. The impregnated support was then coupled to a razor using an adhesive.

1. A microemulsion comprising
an oil phase,
an aqueous phase,
a surfactant, and
a hair modification active.

2. A microemulsion as claimed in claim 1, wherein the oil phase comprises at least one oil selected from polysiloxane, silicone, mineral oil and essential oil.

3. A microemulsion as claimed in claim 1, wherein the surfactant is a non-ionic surfactant.

4. A microemulsion as claimed in claim 3, wherein the surfactant comprises a polyethylene glycol derivative of a triglyceride of a fatty acid.

5. A microemulsion as claimed in claim 1, which further comprises a co-surfactant.

6. A microemulsion as claimed in claim 5, wherein the co-surfactant is a fatty acid ester and/or polyglycerol.

7. A microemulsion as claimed in claim 6, wherein the co-surfactant is a triglyceryl diisostearate.

8. A microemulsion as claimed in claim 1, wherein the depilatory active is selected from one or more of glycerol monothioglycolate, N-acetyl-L-Cysteine, cysteamine potassium thioglycolate and a Bowmann-Birk Inhibitor.

9. A microemulsion as claimed in claim 1, wherein the oil phase comprises 30 to 90 weight % of the microemulsion.

10. A microemulsion as claimed in claim 1, which comprises 5 to 40 weight % water.

11. A microemulsion as claimed in claim 1, which comprises 1 to 30 weight % surfactant.

12. A microemulsion as claimed in claim 1, which additionally comprises a glycol ether.

13. A microemulsion as claimed in claim 1, wherein the droplets of the dispersed phase are less than 200 nm in size.

14. A microemulsion according to claim 1, which further includes at least one skin enhancing agent.

15. A microemulsion according to claim 14, wherein the skin enhancing active comprises alpha hydroxyl acids (AHAs), salicylic acid, glycolic acid, lactic acid, malic acid, citric acid, tartaric acid, trichloracetic acid, a Polyhydroxyacid (PHA), and/or gluconalactone.

16. A microemulsion according to claim 14, wherein the skin enhancing active is present in an amount in the range 0.1 to 2.0 by weight of the composition.

17. A composite for use in a shaving apparatus, which composite comprises
a support, and
the microemulsion of claim 1 deposited on the support.

18. A composite as claimed in claim 17, wherein the microemulsion is impregnated into the support.

19. A composite as claimed in claim 17, wherein the support is formed from a polymer.

20. A shaving apparatus comprising
a shaving head, and
the composite of claim 17, said composite being configured to release at least some of the microemulsion from the shaving apparatus when the apparatus is in use.

21. An apparatus as claimed in claim 20, wherein the shaving head contains at least one blade and wherein the composite is located adjacent the at least one blade.

22. A shaving apparatus which includes:
a shaving head, and
a support member having deposited thereon, or impregnated therein, a microemulsion containing a depilatory active.

23. A shaving apparatus as claimed in claim 22, wherein the support includes PTFE, a porous material, a fibrous material, leather or a cellulose based material.

24. A shaving apparatus comprising
a shaving head, and
means containing the microemulsion of claim 1, said means for releasing at least some of the microemulsion from the shaving apparatus when the apparatus is in use.

25. A method of modifying hair comprising:
applying the microemulsion of claim 1 onto the skin, optionally, in the presence of water,
leaving the microemulsion on the skin for a residence time sufficient to degrade hairs on the skin, and
removing the microemulsion and depilated hairs from the skin.
